(19) United States
(12) Patent Application Publication
(10) Pub. No.: US 2023/0390479 A1
(43) Pub. Date: Dec. 7, 2023

(54) MANUAL SUCTION DEVICE

(71) Applicant: Michael McNally, Ozark, MO (US)

(72) Inventor: Michael McNally, Ozark, MO (US)

(21) Appl. No.: 18/323,270

(22) Filed: May 24, 2023

Related U.S. Application Data
(60) Provisional application No. 63/348,006, filed on Jun. 1, 2022.

(51) Int. Cl. A61M 1/00; A61M 39/24

(52) U.S. Cl. CPC A61M 1/84; A61M 39/24; A61M 1/62

(57) ABSTRACT

A disposable, hand-held, manually operated, and powered suction device for use in both emergent and non-emergent hospital and pre-hospital care settings evacuates media from body cavities created or formed.

### CROSS-REFERENCE TO RELATED APPLICATIONS

This application claims the benefit of U.S. Provisional Application No. 63/348,006 filed on Jun. 1, 2022, which is hereby incorporated in its entirety.

### FIELD OF THE INVENTION

The present invention relates to a disposable, manually powered and operated suction device used to clear human and/or animal airways or body orifices of unwanted media.

### BACKGROUND OF THE INVENTION

Life and death situations arise daily, and medical emergencies occur at any given time. These emergencies do not always happen in a controlled environment, such as a hospital or emergency room. In order to sustain the life of a patient, life saving devices need to be readily available.

Today, there are a variety of suction devices currently on the market. Most devices are fragile and easily broken while utilized with other medical equipment. In addition, the current suction devices require additional assembly and/or are battery operated or require a power source.

Despite the development and advancement in the medical industry, there remains a need to provide a sturdy and durable manual suction device for extracting and collecting bodily fluids and/or media from wounds. As in any technique or device used in the body, special steps must be taken to ensure that the suction pump flows effectively to prevent backflow and does not cause more harm to the patient. The tubing of the suction device also requires frequent cleaning or sterilization to avoid contamination.

Recently, the need for manually powered suction devices have become apparent. First responders, military personnel, and laypersons in austere environments lack access to power sources at any given moment to energize an electrical suction device.

Therefore, there exists a need for a reliable, efficient, manually powered and operated suction device for use in emergent and non-emergent hospital and pre-hospital care settings to evacuate debris and/or media from body cavities.

### SUMMARY OF THE INVENTION

The present invention is directed to a manual suction device that is hand-held, manually operated and powered, and capable of producing strong suction power and pressure for extracting media from a source. More specifically, the device provides a user with an easily accessible device capable of clearing airways and body cavities of large amounts of media such as unwanted fluids, secretions, blood, vomit, air, and/or debris. In addition, the manual suction device is capable of extracting media from a variety of sources, such as eliminating unwanted material in the oral cavity, thoracentesis, air in the chest caused by a tension pneumothorax, ascites in the gut, abdominal paracentesis, urine in the bladder, and foreign objects in open wounds. The device can also be used on various cavities in the body such as the mouth, chest, abdomen, and those created or formed by penetrating objects.

In an exemplary embodiment, the manual suction device for evacuating media from a source comprises a drainage tube, wherein the drainage tube comprises a drain tube first end and a drain tube second end; a pump unit, wherein the pump unit comprises a plunger, a plunger first end, a cylinder, a gasket, and a second outlet; a pump tube, wherein the pump tube comprises of a pump tube first end and pump tube second end; a media collector, wherein the media collector comprises a container, an opening, and a second inlet; a media collector tube, wherein the media collector tube comprises a collector tube first end and a collector tube second end; a connecting mechanism, wherein the connecting mechanism comprises a connector first end, a connector middle end, and a connector second end; and a first check valve and a second check valve; wherein the gasket is permanently attached to the plunger first end; the gasket and the thumb rest are located on opposing ends of the plunger; the plunger is configured to be removably inserted into the cylinder; the pump tube first end is fixedly attached to the connector second end creating a water-tight connection and the pump tube second end is fixedly attached to the second outlet creating a water-tight connection; the drain tube first end is inserted into the source and the drain tube second end is fixedly attached to the first check valve creating a water-tight connection; the connector first end is fixedly attached to the first check valve; the pump tube first end is fixedly attached to the connector second end creating a water-tight connection, and the connector middle end is fixedly attached to the second check valve; and the collector tube first end is fixedly attached to the second check valve creating a water-tight connection and the collector tube second end is fixedly attached to the second inlet creating a water-tight connection.

In one embodiment, the connecting mechanism comprises a “T” connector. In an alternate embodiment, the connecting mechanism comprises a “Y” connector.

In an embodiment, the media comprises air, fluid, secretions, blood, vomit, debris, particulate matter, or any combination thereof.

In yet another embodiment, the connecting mechanism comprises plastic, wood, cork, rubber, metal, bamboo, or any combination thereof.

In an alternate embodiment, the first check valve and the second check valve comprise a chest drain valve.

In one embodiment, the container comprises a disposable, detachable bag.

In yet another embodiment, the source comprises a body cavity, such as oral, chest, or abdomen cavities or a cavity created by an external force such as a projectile, wound, or an infection.

In another embodiment, the pump unit is configured to evacuate the media from the source when the plunger is pulled from the cylinder creating negative pressure throughout the pump unit causing the media to travel from the body cavity into the drainage tube and the pump tube.

In yet another embodiment, the pump unit is configured to deposit the media into the media collector when the plunger is removably inserted into the cylinder creating positive pressure causing the media to flow through the media collector tube and into the container.

In an alternate embodiment, the manual suction device for evacuating media from a source comprises a drainage tube wherein said drainage tube comprises a drain tube first end and a drain tube second end; a pump unit, wherein the pump unit comprises a plunger, a plunger first end, a cylinder, a gasket, and a second outlet; a pump tube, wherein the pump tube comprises pump tube first end and pump tube second end; a media collector, wherein the media collector comprises a container, an opening, and a second inlet; a media collector tube, wherein the media collector tube comprises a collector tube first end and a collector tube second end; a connecting mechanism, wherein the connecting mechanism comprises a connector first end, a connector middle end, and a connector second end; and a first check valve and a second check valve, wherein each check valve comprises a first inlet, a first outlet, and an internal mechanism; wherein the gasket is permanently attached to the plunger first end; the gasket and the thumb rest are located on opposing ends of the plunger; the plunger is configured to be removably inserted into the cylinder; the pump tube first end is fixedly attached to the connector second end creating a water-tight connection and the pump tube second end is fixedly attached to the second outlet creating a water-tight connection; the drain tube first end is inserted into the source and the drain tube second end is securely attached to the first inlet of the first check valve creating a water-tight connection; the connector first end is fixedly attached to the first outlet of the first check valve; the pump tube first end is fixedly attached to the connector second end creating a water-tight connection, and the connector middle end is fixedly attached to the first inlet of the second check valve; and the collector tube first end is fixedly attached to the first outlet of the second check valve creating a water-tight connection and the collector tube second end is fixedly attached to the second inlet creating a water-tight connection.

In one embodiment, the connecting mechanism comprises a “T” connector. In another embodiment, the connecting mechanism comprises a “Y” connector.

In an embodiment, the media comprises air, fluid, secretions, blood, vomit, debris, particulate matter, or any combination thereof.

In yet another embodiment, the connecting mechanism comprises plastic, wood, cork, rubber, metal, bamboo, or any combination thereof.

In an alternate embodiment, the first check valve and the second check valve comprise a chest drain valve.

In yet another embodiment, the container comprises a disposable, detachable bag.

In an alternate embodiment, the source comprises a body cavity, such as oral, chest, or abdomen cavities or a cavity created by an external force such as a projectile, wound, or an infection.

In one embodiment, the pump unit is configured to evacuate the media from the source when the plunger is pulled from the cylinder creating negative pressure throughout the pump unit causing the media to travel from the body cavity into the drainage tube and the pump tube.

In the same embodiment, the pump unit is configured to deposit the media into the media collector when the plunger is removably inserted into the cylinder creating positive pressure causing the media to flow through the media collector tube and into the container.

Various objects, features, aspects, and advantages of the inventive subject matter will become apparent form the following detailed description of exemplary embodiments, along with the accompanying figures in which like numerals represent like components.

### BRIEF DESCRIPTION OF DRAWINGS

FIG. 1 depicts an exemplary embodiment of a manual suction device.

FIG. 2 depicts an exemplary embodiment of a pump unit.

FIG. 3A depicts an exemplary embodiment of a check valve.

FIG. 3B depicts an alternate embodiment of a connecting mechanism utilized in a manual suction device.

FIG. 4 depicts an exemplary embodiment of a manual suction device while in use.

### DETAILED DESCRIPTION OF THE INVENTION

The following description is not an admission that any of the information provided herein is prior art or relevant to the present invention, or that any publication specifically or implicitly referenced is prior art. Any publications cited in the description are incorporated by reference herein. Where a definition or use of a term in an incorporated reference is inconsistent or contrary to the definition of that term provided herein, the definition of that term provided herein applies and the definition of that term in the reference does not apply.

As used in the description herein and throughout the claims that follow, the meaning of “a,” “an,” and “the” includes plural reference unless the context clearly dictates otherwise. Also, as used in the description herein, the meaning of “in” includes “into” and “on” unless the context clearly dictates otherwise.

As used herein, the term “about” in conjunction with a numeral refers to a range of that numeral starting from 10% below the absolute value of the numeral to 10% above the absolute value of the numeral, inclusive.

As used in this description, the term “media” includes but is not limited to: air, unwanted fluids, secretions, blood, vomit, debris, particulate matter, or any combination thereof.

As used in this description, the terms “manual suction device” and “device” are used interchangeably.

The present invention is directed to a manual suction device that is hand-held, manually operated and powered, and capable of producing strong suction power and pressure for extracting media from a source. More specifically, the device provides a user with an accessible device capable of clearing airways and body cavities of large amounts of media such as unwanted fluids, secretions, blood, vomit, air, and/or debris. In addition, the manual suction device is capable of extracting media from a variety of sources, such as eliminating unwanted material in the oral cavity, thoracentesis, air in the chest caused by a tension pneumothorax, ascites in the gut, abdominal paracentesis, urine in the bladder, and foreign objects in open wounds. The device can also be used on various cavities in the body such as oral, chest, abdomen, and those created or formed by penetrating objects.

The manual suction device is configured to be used in both medical and non-medical settings by the military, emergency medical technicians, or any other person familiar with the device in the field or in austere environments where electrical power or batteries are not always readily available. More specifically, the manual suction device can be used where one-directional suction is desired in order to convey media upwards.

The manual suction device is configured to enable a user to clear many forms of debris and/or media in a patient, human or animal, in a short amount of time permitting rapid emergency treatment and transportation of patients. Media, such as fluids, secretions, blood, vomit, air, and/or debris, is evacuated from its source to a media collector by means of a pump unit by providing positive and negative pressure in a cyclic motion thereby moving the media through one-way valves into a container. This enables a user to collect or direct media (e.g., blood) easily after suctioned, without risk of contamination.

The manual suction device provides a durable, lightweight, and economical way of collecting and analyzing media, while also ensuring the media drawn from the source remains isolated in the media collector, protecting the patient from additional risk or harm.

The manual suction device comprises a plurality of check valves configured to move media through the tubes in only one direction and into the pump unit, thus transferring media in a controlled environment while minimizing waste and ensuring no contamination enters back into the source.

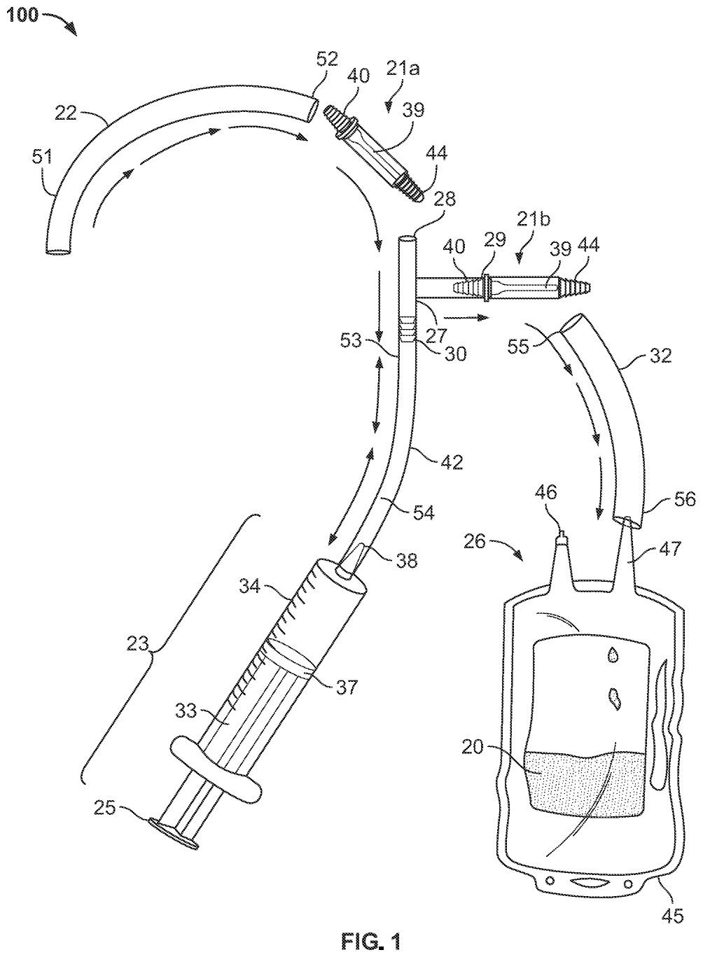

Exemplary configurations of the present invention are depicted in FIGS. 1 to 4, in which manual suction device 100 is configured to clear airways and body cavities of large amounts of media such as unwanted fluids, secretions, blood, vomit, air, and/or debris.

FIG. 1 depicts an exemplary embodiment of manual suction device 100, wherein device 100 comprises pump unit 23, media collector 26, connecting mechanism 27, and a plurality of check valves 21. In one embodiment, plurality of valves 21 comprises valve 21a and valve 21b. In one embodiment, pump unit 23 comprises plunger 33, cylinder 34, gasket 37, plunger first end 57, second outlet 38, thumb rest 25, and pump tube 42; wherein gasket 37 and thumb rest 25 are located on opposite ends of plunger 33, gasket 37 is permanently attached to plunger first end 57, and plunger 33 is configured to be removably inserted into cylinder 34. In an embodiment, gasket 37 is configured to provide an air-tight seal when inserted into cylinder 34.

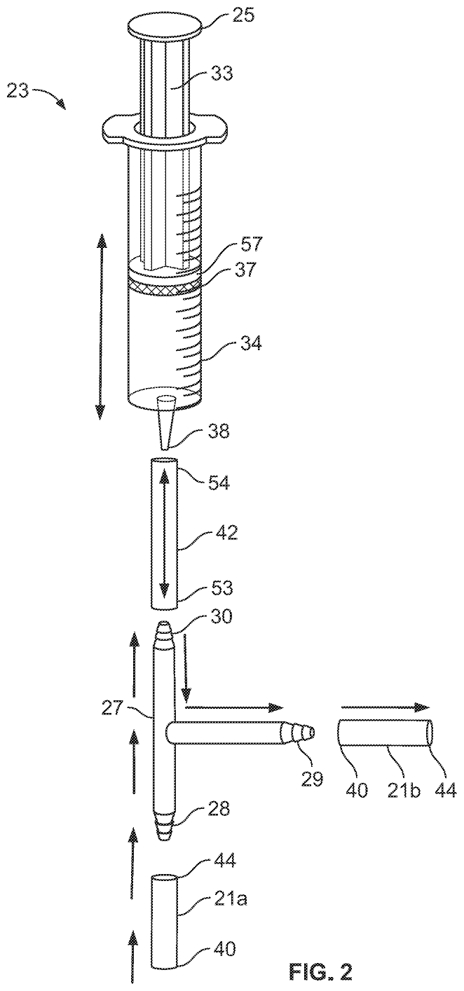

In an embodiment, pump tube 42 comprises pump tube first end 53 and pump tube second end 54, wherein pump tube first end 53 is securely attached to connector second end 30 of connecting mechanism 27 and pump tube second end 54 is securely attached to second outlet 38 creating a water-tight seal at both connection points (see FIG. 2).

In an exemplary embodiment, manual suction device 100 comprises a plurality of check valves 21 that are configured to allow media 20 to flow in one direction only. In an embodiment, media 20 flows through drainage tube 22 through first inlet 40 into pump tube 42 where it is prevented from reentering drainage tube 22 by internal mechanism 39.

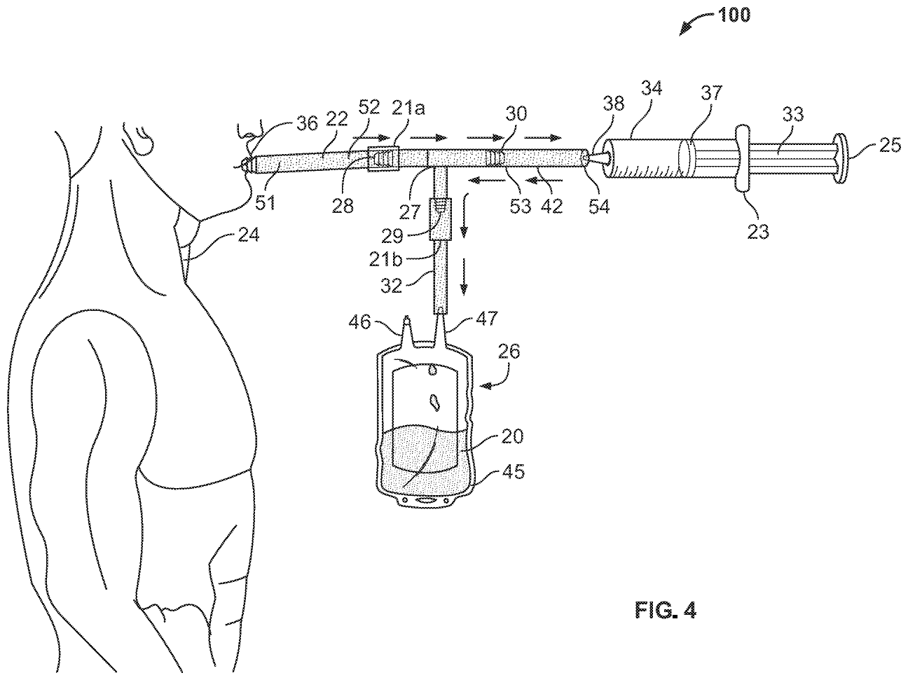

In an embodiment, check valve 21 is configured to accept fluid and media 20 through first inlet 40 and configured to prohibit fluid and media 20 from flowing back into drainage tube 22 through first outlet 44 (see FIG. 1). Drainage tube 22 comprises drain tube first end 51 and drain tube second end 52, wherein drain tube second end 52 is fixedly attached to first inlet 40 on first check valve 21a. Drain tube first end 51 is configured to be inserted into a body cavity such as the mouth to extract media 20. Negative pressure is created by pump unit 23 thereby extracting unwanted materials from source 24 or body cavity 36 (see FIG. 4).

In an embodiment, connecting mechanism 27 comprises connector first end 28, connector middle end 29, and connector second end 30; wherein connector first end 28 is fixedly attached to first outlet 44 of first check valve 21a, connector second end 30 is securely inserted into pump tube first end 53, and connector middle end 29 is fixedly attached to first inlet 40 of second check valve 21b.

In an exemplary embodiment, media collector 26 comprises container 45, opening 46, second inlet 47, and media collector tube 32; wherein media collector tube 32 comprises collector tube first end 55 and collector tube second end 56. In this embodiment, collector tube first end 55 is securely attached to first outlet 44 of second check valve 21b and collector tube second end 56 is securely attached to second inlet 47 creating a water-tight connection. In this embodiment, container 45 is configured to accept unwanted media 20 being suctioned from drainage tube 22 through first check valve 21a, connecting mechanism 27, second check valve 21b, and media collector tube 32 into second inlet 47. In an embodiment, opening 46 is configured to release pressure in container 45. In one embodiment, opening 46 is a pressure release valve.

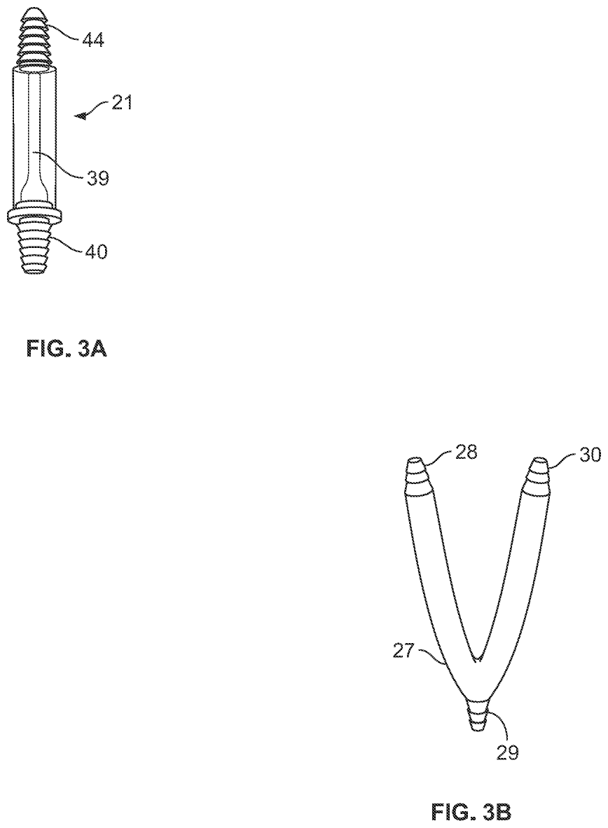

In one embodiment, connecting mechanism 27 comprises a “T” connector (see FIG. 2). In an alternate embodiment, connecting mechanism 27 comprises a “Y” connector (see FIG. 3B).

In an embodiment, pump unit 23 activates when a user gently pulls thumb rest 25 on plunger 33 from cylinder 34 creating negative pressure through drainage tube 22 and pump tube 42 causing media 20 from a person's body cavity 36 (see FIG. 4) to be drawn through drainage tube 22, into connecting mechanism 27, through pump tube 42 and into cylinder 34 of pump unit 23. In this embodiment, thumb rest 25 on pump unit 23 is then compressed, pushing plunger 33 and gasket 37 forward towards second outlet 38 creating positive pressure, allowing media 20 to flow from pump tube 42 through connector second end 30 into media collector tube 32 and finally into container 45 of media collector 26.

FIG. 2 depicts an exemplary embodiment of pump unit 23 and connecting mechanism 27. In an embodiment, pump unit 23 comprises plunger 33, plunger first end 57, thumb rest 25, cylinder 34, gasket 37, second outlet 38, and pump tube 42; wherein gasket 37 is permanently attached to plunger first end 57, wherein gasket 37 and thumb rest 25 are on opposite ends of plunger 33, and plunger 33 is configured to be removably inserted into cylinder 34. In an embodiment, pump tube 42 comprises pump tube first end 53 and pump tube second end 54, wherein pump tube first end 53 is fixedly attached to connector second end 30 and pump tube second end 54 is fixedly attached to second outlet 38, thus creating a water-tight connection on both ends of pump tube 42. In one embodiment, material of construction for pump tube 42, drainage tube 22, and media collector tube 32 comprises durable, disposable tubing.

In an embodiment, connecting mechanism 27 comprises connector first end 28, connector middle end 29, and connector second end 30; wherein pump tube first end 53 is fixedly attached to connector second end 30, first inlet 40 of second check valve 21b is fixedly attached to connector middle end 29, and first outlet 44 of first check valve 21a is fixedly attached to connector first end 28 thereby creating a water-tight at all three connection points. In the same embodiment, pump tube second end 54 is fixedly attached to second outlet 38 creating a water-tight seal at the connection point.

In an exemplary embodiment, pump unit 23 comprises a disposable standard syringe.

In one embodiment, cylinder 34 is transparent and configured to enable a user to easily view and assess the media and/or matter extracted from source 24.

In an embodiment, source 24 comprises a body cavity, such as oral, chest, or abdomen cavities or a cavity created by an external force such as a projectile, wound, or an infection.

In an exemplary embodiment, pump unit 23 comprises plunger 33 configured to guide air in and out of manual suction device 100.

FIG. 3A depicts an exemplary embodiment of check valve 21. In an exemplary embodiment, check valve 21 comprises first inlet 40, internal mechanism 39, and first outlet 44; wherein internal mechanism 39 is located between first inlet 40 and first outlet 44 and is configured to allow movement of media 20 in one direction only.

In one embodiment, internal mechanism 39 comprises of a fluid-impervious material. In another embodiment, internal mechanism 39 comprises of a ball and O-ring. In yet another embodiment, internal mechanism 39 comprises of a flap hinged on one side.

FIG. 4 depicts an exemplary embodiment of manual suction device 100 in use. In this embodiment, unwanted media 20 is removed from source 24, by pulling and pushing thumb rest 25 of plunger 33 in pump unit 23. Pulling thumb rest 25 from cylinder 34 creates negative pressure and forces first check valve 21a into an open position thereby extracting fluids and matter from source 24. In the alternative, pushing thumb rest 25 into cylinder 34 creates positive pressure and forces first check valve 21a to close and second check valve 21b to open thereby pushing extracted fluids and matter into media collector 26. Alternating push and pull movements on plunger 33 of pump unit 23 creates a cyclic movement of media from source 24 to media collector 26.

In an exemplary embodiment, drainage tube 22 comprises drain tube first end 51 and drain tube second end 52, wherein drain tube first end 51 is inserted into source 24 or body cavity 36 and drain tube second end 52 is fixedly attached to first inlet 40 of first check valve 21a creating a water-tight connection. First check valve 21a is fixedly attached between drainage tube 22 and connector first end 28 of connecting mechanism 27.

In another embodiment, pump tube 42 comprises pump tube first end 53 and pump tube second end 54, wherein pump tube first end 53 is fixedly attached to connector second end 30 and pump tube second end 54 is fixedly attached to second outlet 38 creating a water-tight connection. Second check valve 21b is fixedly attached between connector middle end 29 and collector tube first end 55.

In an embodiment, first check valve 21a and second check valve 21b are configured to allow air and fluid to flow in one direction only. FIGS. 1 and 4 depict the fluid flow with arrows. For example, first check valve 21a is configured to allow fluid to flow from drainage tube 22 into connector first end 28 and prevent fluid from flowing in the opposite direction from connector first end 28 to drainage tube 22. In the same embodiment, second check valve 21b is configured to allow fluid to flow from connector middle end 29 into media collector tube 32 and prevent fluid flow in the opposite direction of media collector tube 32 into connector middle end 29.

In one embodiment, material of construction for connecting mechanism 27 comprises plastic, wood, cork, rubber, metal, bamboo, or any combination thereof.

In an exemplary embodiment, pump unit 23 is manually operated. In one embodiment, plunger 33 of pump unit 23 is compressed using one hand. In an alternate embodiment, plunger 33 of pump unit 23 is compressed using two hands.

In an exemplary embodiment, pump unit 23 comprises medical grade components. In one embodiment, pump unit 23 is a single use applicator and is disposable after use. Drainage tube 22 and pump unit 23 are intended to be used in, on, or in contact with the body. If additional drainage is thereafter desired, a new clean or sterile pump unit 23 may be attached and activated as described above.

Thus, specific embodiments of manual suction device 100 and method of using device 100 have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

1. A manual suction device for evacuating media from a source, comprising:
(a) a drainage tube comprising a drain tube first end and a drain tube second end;
(b) a pump unit comprising a plunger, a plunger first end, a cylinder, a gasket, and a second outlet;
(c) a pump tube comprising pump tube first end and pump tube second end;
(d) a media collector comprising a container, an opening, and a second inlet;
(e) a media collector tube comprising a collector tube first end and a collector tube second end;
(f) a connecting mechanism comprising a connector first end, a connector middle end, and a connector second end; and
(g) a first check valve and a second check valve;
wherein:
i. said gasket is permanently attached to said plunger first end;
ii. said gasket and said thumb rest are located on opposing ends of said plunger;
iii. said plunger is configured to be removably inserted into said cylinder;
iv. said pump tube first end is fixedly attached to said connector second end creating a water-tight connection and said pump tube second end is fixedly attached to said second outlet creating a water-tight connection;
v. said drain tube first end is inserted into said source and said drain tube second end is fixedly attached to said first check valve creating a water-tight connection;
vi. said connector first end is fixedly attached to said first check valve;
vii. said pump tube first end is fixedly attached to said connector second end creating a water-tight connection, and said connector middle end is fixedly attached to said second check valve; and
viii. said collector tube first end is fixedly attached to said second check valve creating a water-tight connection and said collector tube second end is fixedly attached to said second inlet creating a water-tight connection.

2. The manual suction device as recited in claim 1, wherein said connecting mechanism comprises a “T” connector.

3. The manual suction device as recited in claim 1, wherein said connecting mechanism comprises a “Y” connector.

4. The manual suction device as recited in claim 1, wherein said media comprises air, fluid, secretions, blood, vomit, debris, particulate matter, or any combination thereof.

5. The manual suction device as recited in claim 1, wherein said connecting mechanism comprises plastic, wood, cork, rubber, metal, bamboo, or any combination thereof.

6. The manual suction device as recited in claim 1, wherein said first check valve and said second check valve comprise a chest drain valve.

7. The manual suction device as recited in claim 1, wherein said container comprises a disposable, detachable bag.

8. The manual suction device as recited in claim 1, wherein said source comprises a body cavity, such as oral, chest, or abdomen cavities or a cavity created by an external force such as a projectile, wound, or an infection.

9. The manual suction device as recited in claim 1, wherein said pump unit is configured to evacuate said media from said source when said plunger is pulled from said cylinder creating negative pressure throughout said pump unit causing said media to travel from said body cavity into said drainage tube and said pump tube.

10. The manual suction device as recited in claim 9, wherein said pump unit is configured to deposit said media into said media collector when said plunger is removably inserted into said cylinder creating positive pressure causing said media to flow through said media collector tube and into said container.

11. A manual suction device for evacuating media from a source, comprising:
(a) a drainage tube comprising a drain tube first end and a drain tube second end;
(b) a pump unit comprising a plunger, a plunger first end, a cylinder, a gasket, and a second outlet;
(c) a pump tube comprising pump tube first end and pump tube second end;
(d) a media collector comprising a container, an opening, and a second inlet;
(e) a media collector tube comprising a collector tube first end and a collector tube second end;
(f) a connecting mechanism comprising a connector first end, a connector middle end, and a connector second end; and
(g) a first check valve and a second check valve, each comprising a first inlet, a first outlet, and an internal mechanism;
wherein:
i. said gasket is permanently attached to said plunger first end;
ii. said gasket and said thumb rest are located on opposing ends of said plunger;
iii. said plunger is configured to be removably inserted into said cylinder;
iv. said pump tube first end is fixedly attached to said connector second end creating a water-tight connection and said pump tube second end is fixedly attached to said second outlet creating a water-tight connection;
v. said drain tube first end is inserted into said source and said drain tube second end is securely attached to said first inlet of said first check valve creating a water-tight connection;
vi. said connector first end is fixedly attached to said first outlet of said first check valve;
vii. said pump tube first end is fixedly attached to said connector second end creating a water-tight connection, and said connector middle end is fixedly attached to said first inlet of said second check valve; and
viii. said collector tube first end is fixedly attached to said first outlet of said second check valve creating a water-tight connection and said collector tube second end is fixedly attached to said second inlet creating a water-tight connection.

12. The manual suction device as recited in claim 11, wherein said connecting mechanism comprises a “T” connector.

13. The manual suction device as recited in claim 11, wherein said connecting mechanism comprises a “Y” connector.

14. The manual suction device as recited in claim 11, wherein said media comprises air, fluid, secretions, blood, vomit, debris, particulate matter, or any combination thereof.

15. The manual suction device as recited in claim 11, wherein said connecting mechanism comprises plastic, wood, cork, rubber, metal, bamboo, or any combination thereof.

16. The manual suction device as recited in claim 11, wherein said first check valve and said second check valve comprise a chest drain valve.

17. The manual suction device as recited in claim 11, wherein said container comprises a disposable, detachable bag.

18. The manual suction device as recited in claim 11, wherein said source comprises a body cavity, such as oral, chest, or abdomen cavities or a cavity created by an external force such as a projectile, wound, or an infection.

19. The manual suction device as recited in claim 11, wherein said pump unit is configured to evacuate said media from said source when said plunger is pulled from said cylinder creating negative pressure throughout said pump unit causing said media to travel from said body cavity into said drainage tube and said pump tube.

20. The manual suction device as recited in claim 19, wherein said pump unit is configured to deposit said media into said media collector when said plunger is removably inserted into said cylinder creating positive pressure causing said media to flow through said media collector tube and into said container.
